(19) United States
(12) Patent Application Publication
(10) Pub. No.: US 2025/0000493 A1
(43) Pub. Date: Jan. 2, 2025

(54) TOOTHBRUSH WITH A SAMPLE COLLECTION ASSEMBLY

(71) Applicant: Binx Health Limited, Trowbridge (GB)

(72) Inventor: Jeffrey Luber, Cambridge, MA (US)

(21) Appl. No.: 18/709,306

(22) PCT Filed: Nov. 14, 2022
(86) PCT No.: PCT/IB2022/000758

§ 371 Date: May 10, 2024

Related U.S. Application Data
(60) Provisional application No. 63/279,009, filed on Nov. 12, 2021.

(51) Int. Cl. A61B 10/02; A46B 17/08; A61B 10/00

(52) U.S. Cl. CPC A61B 10/02; A46B 17/08; A61B 10/0051; A61B 10/0096; A46B 2200/1066

(57) ABSTRACT

The invention provides a toothbrush including a sample collection member operably associated therewith and configured to collect a biological sample of interest from a user's mouth or other orifice. The sample collection member is either integrally formed with or releasably attached to a portion of the toothbrush. Accordingly, the toothbrush may be utilized as a vehicle for accessing a user's mouth and collecting, via the sample collection member, a biological sample of interest, such as saliva, cellular material within the mouth (i.e., check cells), and/or respiratory mucosa. Upon collection of a biological sample, either the entire unit (toothbrush and sample collection member) or simply the sample collection member can be shipped to a laboratory for processing or can be used for testing at home.

### TECHNICAL FIELD

The invention relates generally to sample collection, and, more particularly, to a toothbrush including a sample acquisition assembly for collecting a biological sample of interest from a user.

### BACKGROUND

A critical aspect of diagnostic testing involves the initial step of collecting a sample. In most instances, collection of a biological sample is performed in a medical setting. It is important that the integrity of the sample be maintained and contamination prevented. In recent years, however, self-tests and at-home diagnostics have become increasingly popular. In particular, it has become more common for patients to collect a biological sample at home and then either provide the sample to a laboratory for subsequent testing or simply utilize an at-home diagnostic device to process the sample. However, conventional collection devices, such as a typical sample tube (i.e., Eppendorf or the like) have drawbacks and present certain difficulties during use. Such difficulties include, but are not limited to, awkward shape and size, unwieldy numbers of steps for use, unclear indications of orientation of tube, instability on flat surface, caps resistant to closure, inconsistent combining of sample with stabilization composition, and the like.

### SUMMARY

The present invention provides a sample collection apparatus in the form of a toothbrush with which a sample collection member is associated. The sample collection member includes a receptacle in which a biological sample of interest is collected. The sample collection member is either integrally formed with the toothbrush or releasably attached to a portion of the toothbrush. While the toothbrush is in fact functional (i.e., it can be used to brush the user's teeth), the toothbrush provides a vehicle for accessing the user's mouth, or other orifice, and allows for collection of a biological sample via the sample collection member. The biological sample of interest may include, but is not limited to, saliva, cellular material within the mouth (i.e., cheek cells), and/or respiratory mucosa. Upon collection of a biological sample, either the entire unit (toothbrush and sample collection member integrally formed therewith) or simply the sample collection member can be shipped to a laboratory for processing or can be used for testing at home. Devices of the invention are useful for repeated sample collection. For example, a device of the invention may have a detachable sample collection member that is used to acquire sample, after which the sample collection member is removed and used for testing. The toothbrush portion of the device can continue to be used as a toothbrush and another sample acquisition member can be added when necessary to collect additional sample. Accordingly, the present invention improves self-testing and at-home diagnostics by providing a simple, yet effective, mechanism for collecting biological samples.

### DETAILED DESCRIPTION

By way of overview, the present invention provides a toothbrush comprising a sample acquisition assembly for collecting a biological sample of interest from. The toothbrush is generally in the form of a conventional toothbrush, and includes a handle portion providing an area to be gripped by a user and a head portion which includes bristles to engage a user's teeth during use.

The toothbrush further includes a sample collection member adapted to receive and retain a biological sample therein. In particular, the sample collection member includes a receptacle or chamber having an interior volume for receiving and retaining a biological sample within. In some embodiments, the sample collection member is integrally formed with a portion of the toothbrush. For example, the sample collection member may form part of the handle portion and/or head portion of the toothbrush. It should be noted, however, that in other embodiments, the sample collection member may be releasably attached to the toothbrush. In either instance, the receptable may be provided adjacent to the head portion of the toothbrush to thereby allow for collection of a biological sample within. For example, when attempting to collect a biological sample, the user need only to place the toothbrush within their mouth, or other orifice, and collect the desired sample. The biological sample may include a bodily fluid, such as saliva. As such, a user need only place the toothbrush in their mouth and simply excrete saliva into the receptable. In some embodiments, the sample collection member further includes a collection swab member configured to extend from and retract within an interior volume of the receptacle. For example, the toothbrush may allow for some form of user input resulting in extension and retraction of the swab member. Such user input may include a mechanical means, such as a slidable rail system or the like. As such, incorporation of a swab member allows for collection of at least one of a nasopharyngeal sample and cheek sample, for example.

The receptable may further include a lid configured to selectively transition between an open position and a closed position. When the lid is in the open position, the interior volume of the receptable is uncovered, thereby allowing for a biological sample to be collected within. When the lid is in the closed position, the interior volume is covered and the lid retains the biological sample within the receptacle.

Upon collection of a biological sample, the user may then provide the sample for testing. For example, in the event that the toothbrush and sample collection member are integrally formed with one another, the entire unit may be provided to a laboratory for testing, and the results can be returned to the user. In the event that the sample collection member is releasably attached to the toothbrush, a user need only detach and separately provide the sample collection member to a laboratory for testing. In such an embodiment, the user can retain the toothbrush and utilize it for normal teeth brushing activities. In the event that additional sample collection is needed, a user can simply attach a new and unused sample collection member to the same toothbrush and collect the desired sample. In either instance, the toothbrush provides a vehicle in which a user can perform certain collection methods that would otherwise be difficult to replicate at home.

In some embodiments, the sample collection member is an abrasive or semi-abrasive surface that engages with, for example, a subject's check to cause release of cells for analysis. In other embodiments, the sample collection member extends from the handle of the toothbrush by, for example, a push button mechanism or a slidable controller that causes the sample collection member to slidably release over the terminus of the toothbrush. In some instances, the sample collection member is released from the toothbrush after sample acquisition and is processed at the point of care or at a laboratory (if the sample collection member or the sample itself is shipped from the point of collection). As discussed above, the sample collection member can collect saliva, which is then expelled from the sample collection member into an apparatus for testing or shipping. Finally, in some embodiments, the sample collection member is releasably attached or integrated with a water pick, flossing device or other device that accesses the oral cavity.

Additional advantages and alternatives of the invention are apparent based on the disclosure presented herein.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A sample collection assembly comprising:
a toothbrush; and
a sample collection member operably associated with the toothbrush.

2. The assembly of claim 1, wherein the sample collection member is integrally associated with the toothbrush or releasably attached to a portion of the toothbrush.

3. The assembly of claim 2, wherein the sample collection member comprises a receptacle associated with at least one of a handle portion and a head portion of the toothbrush.

4. The assembly of claim 3, wherein the receptacle comprises an interior volume shaped and/or sized for receiving and retaining a biological sample therein.

5. The assembly of claim 4, wherein the biological sample comprises fluid and/or tissue collected from a user's mouth during use of the toothbrush.

6. The assembly of claim 5, wherein the biological sample comprises saliva and/or cellular material.

7. The assembly of claim 2, wherein the receptable further comprises a lid configured to selectively transition between an open position and a closed position.

8. The assembly of claim 7, wherein, when the lid is in the open position, a biological sample can be collected within an interior volume of the receptacle, and, when the lid is in the closed position, the lid retains the biological sample within the interior volume of the receptacle.

9. The assembly of claim 2, wherein the sample collection member further comprises a collection swab member configured to extend from and retract within an interior volume of the receptacle.

10. The assembly of claim 9, wherein the swab is configured to provide collection of at least one of a nasopharyngeal sample and cheek sample.

11. The assembly of claim 3, wherein the sample collection member is releasably attached to at least one of a handle portion and a head portion of the toothbrush.

12. The assembly of claim 11, wherein the sample collection member is releasably attached via a snap fit or pressure fit means.
